SEQUENCE LISTING (6 sequences)

SEQ ID NO: 1 - DNA, 2959 bp, Oryza sativa
  CDS (204)..(2636)
  mutation (2013)..(2013): SNP3
gcgcacgctttacaggtggctcccccgccgtctcccccgcgtgccctcctcgccgtcgtcgctcgctgcccatttcatcccccgcacggcacgcgcgggacgaaagcaaagcacgcgcacacactcaaacccgcgcctcctccgctctcgtccgtcgcttcggttcggttccggctcgatcggggaggttcctcggggcgaccatgtcgtcggccgtcgtcgcgtcatccaccacgttcctcgtcgcgctcgcctcttcggcgtcacggggagggcccaggagggggagggtggtgggcgtggccgcgccgccggccctgctttacgacggccgcgccggaaggctagccctacgggcgccgcctccgccccggcctcggcctcggcgtcgggatgcgggcgtggtgcgccgcgcggatgatggggagaacgaggcggcggtggagcgggcgggtgaggacgacgacgaggaggaggagttctcttcgggcgcgtggcagccgccccgctcgcgtcgcggcggcgtcggcaaggtcttgaagcggaggggcaccgtcccgcccgtcggccggtacggctccggcggtgatgcggcgagagtgcgcggggcggccgcgccggcgccggcgccgacacaggacgccgcctcaagtaagaacggagcgcttctcagcggccgcgacgacgacacacctgcctcacggaacggatcggtcgttaccggcgccgacaagcctgccgccgccacgccgccggtgaccataacgaagctcccagcgccggactcccccgtgatccttccatccgtagacaagccgcagccggagttcgtcatcccagacgcgacggcgccggcgccgccaccgcccggttcaaatcccaggtcgtccgctcctctccccaagcctgacaattcggaatttgcagaggataagagcgcaaaagttgttgagagtgctccgaagccaaaggcgactagatcttcccctattcctgcggtagaagaggagacgtgggatttcaagaaatattttgatctgaacgaaccggacgccgcggaggatggcgatgacgatgatgactgggctgattcagatgcgtcagattctgagatcgaccaggatgacgattcgggccctttggctggggagaatgtcatgaacgtgatcgtggtggctgctgaatgttctccctggtgcaaaacaggtgggcttggagatgttgcaggtgctttacccaaagctttggcgaggagaggacatcgtgttatggttgtggtaccaaggtacggtgattacgcggaagcccaggatgtaggaatcaggaaatactacaaggctgctggacaggatctggaagtgaaatatttccatgcatttatcgatggagttgattttgtgttcattgacgctcctctcttccgtcaccgtcaggatgacatctatggggggaacagacaggaaatcatgaagcgcatgattctgttttgtaaggctgctgttgaggttccttggcacgttccatgcggtggtgtgccctatggggatggcaacttggtgttccttgcaaacgattggcacactgcactcctgcctgtttatctgaaggcatattacagagacaatggcatgatgcagtacactcgctctgtccttgtgatacataatatcgcttaccagggccgtggcccagtagatgaattcccctacatggaattgccggagcactacctggatcacttcaagctgtacgaccccgtcggcggcgagcacgccaacatcttcggcgcgggcctgaagatggcggaccgggtggtgaccgtgagccccggctacctctgggagctgaagacgacggagggcggctggggcctccacgacatcatacgggagaacgactggaagatgaacggcatcgtgaacggcatcgactaccgggagtggaacccggaggtggacgtgcacctgcagtccgacggctacgccaactacaccgtggcctcgctggactccagcaagccgcggtgcaaggcggcgctgcagcgcgagctggggctggaggtgcgcgacgacgtgccgctgatcgggttcatcgggcggctcgacgggcagaaaggtgtggacatcatcggcgacgcgatgccgtggatcgccgggcaggacgtgcagctggtgctgctgggctccggccgccgcgacctggaggtgatgctgcagcggttcgaggcgcagcacaacagcaaggtgcgcgggtgggtggggttctcggtgaagatggcgcaccggatcacggcgggcgccgacgtgctggtcatgccgtcgcggttcgagccgtgcggcctcaaccagctctacgccatggcgtacggcaccgtccccgtcgtgcacgccgtcggcgggctgagggacaccatgtcggcgttcgacccgttcgaggacaccggcctcgggtggacgttcgaccgcgccgagccgcacaagctcatcgaggcgctcggccactgcctcgagacgtaccgcaagtacaaggagagctggagggggctccaggtgcgcggcatgtcgcaggacctcagctgggaccacgccgccgagctctacgaggaggtccttgtcaaggccaagtaccaatggtgaagagaccatgtccgcccgcggtctccgcctgcgcgttcggcagctatagctatagcctccctgaagaagcttggcgctgctgtggaggtgcgactcggtggcggtgtggcgccggcgccggcgcttgcaggggaggtgtttgtgctgaccgagctgtgcgttgaggtgcgcaaattagcttccggtttgtgtgttcatggcgaggcatggtggagttgagggagtattaagctttgtttcagatgcatcatgcatgcgtaataggatgttgtatttaataatgtgtttgttatgtccattgatgcgatgttacttcttcatta

SEQ ID NO: 2 - protein, 810 aa, Oryza sativa
MSSAVVASSTTFLVALASSASRGGPRRGRVVGVAAPPALLYDGRAGRLALRAPPPPRPRPRRRDAGVVRRADDGENEAAVERAGEDDDEEEEFSSGAWQPPRSRRGGVGKVLKRRGTVPPVGRYGSGGDAARVRGAAAPAPAPTQDAASSKNGALLSGRDDDTPASRNGSVVTGADKPAAATPPVTITKLPAPDSPVILPSVDKPQPEFVIPDATAPAPPPPGSNPRSSAPLPKPDNSEFAEDKSAKVVESAPKPKATRSSPIPAVEEETWDFKKYFDLNEPDAAEDGDDDDDWADSDASDSEIDQDDDSGPLAGENVMNVIVVAAECSPWCKTGGLGDVAGALPKALARRGHRVMVVVPRYGDYAEAQDVGIRKYYKAAGQDLEVKYFHAFIDGVDFVFIDAPLFRHRQDDIYGGNRQEIMKRMILFCKAAVEVPWHVPCGGVPYGDGNLVFLANDWHTALLPVYLKAYYRDNGMMQYTRSVLVIHNIAYQGRGPVDEFPYMELPEHYLDHFKLYDPVGGEHANIFGAGLKMADRVVTVSPGYLWELKTTEGGWGLHDIIRENDWKMNGIVNGIDYREWNPEVDVHLQSDGYANYTVASLDSSKPRCKAALQRELGLEVRDDVPLIGFIGRLDGQKGVDIIGDAMPWIAGQDVQLVLLGSGRRDLEVMLQRFEAQHNSKVRGWVGFSVKMAHRITAGADVLVMPSRFEPCGLNQLYAMAYGTVPVVHAVGGLRDTMSAFDPFEDTGLGWTFDRAEPHKLIEALGHCLETYRKYKESWRGLQVRGMSQDLSWDHAAELYEEVLVKAKYQW

SEQ ID NO: 3 - DNA, 21 bp, Artificial sequence
  Synthesized
ggttgggcaggccagcgtatc

SEQ ID NO: 4 - DNA, 21 bp, Artificial sequence
  Synthesized
ccaatgcctaaagagaggtta

SEQ ID NO: 5 - DNA, 17 bp, Oryza sativa
gtcgctcgctgcccatt

SEQ ID NO: 6 - DNA, 16 bp, Oryza sativa
tgcccctccgcttcaa

(19) United States
(12) Patent Application Publication
(10) Pub. No.: US 2008/0201807 A1
(43) Pub. Date: Aug. 21, 2008

(54) Gelatinization Temperature Manipulation

(75) Inventors: Robert James Henry, New South Wales (AU); Daniel Lex Ean Waters, New South Wales (AU)

Correspondence Address: CATALYST LAW GROUP, APC, 9710 SCRANTON ROAD, SUITE S-170, SAN DIEGO, CA, 92121, US

(73) Assignees: SOUTHERN CROSS UNIVERSITY, Lismore (AU); Rural Industries Research and Development Corporation, Barton (AU)

(21) Appl. No.: 11/815,919

(22) PCT Filed: Feb. 13, 2006
(86) PCT No.: PCT/AU2006/000188

§ 371 Date: Aug. 9, 2007

(30) Foreign Application Priority Data
Feb. 11, 2005 (AU) 2005900645
Feb. 13, 2006 (AU) PCT/AU2006/000188

(51) Int. Cl. A01H 5/00; C12N 15/82; C12Q 1/68; A23L 1/00; C12N 9/00

(52) U.S. Cl. 800/312; 435/468; 800/298; 800/320; 800/320.2; 800/320.1; 800/320.3; 800/313; 426/618; 435/183; 435/6

(57) ABSTRACT

The subject invention relates to plants and the starch produced by plants. In particular, the invention relates to processes for the modification of a plant so that the starch produced by the plant comprises amylopectin of an altered structure and/or the starch has an altered gelatinization temperature. The processes principally comprise engineering gene involved in amylopectin synthesis. In a preferred embodiment the starch synthase IIa gene is modified. Also disclosed are processes for altering genes to provide plant which produce starch having a desired gelatinization temperature, and methods of identifying the changes that can be made in genes to achieve a desired gelatinization temperature in the starch produced by a plant comprising such genes. Further disclosed are modified plants and the starch products of the plants.

### TECHNICAL FIELD

The invention described herein relates to plants from which starch is derived. Particularly, the invention relates to the manipulation of the gelatinization temperature of starch as a way of attaining a product comprising starch that has desired properties. It will be appreciated that the scope of the invention is not necessarily limited to the foregoing.

### BACKGROUND ART

Starch is one of the world's most important agricultural products (Fitzgerald, 2004). The bulk of the annual global production of starch is used in food where it is a major source of energy but it is also used in many other non-food applications including industrial uses. For example, starch is used as an adhesive, a coating, in pharmaceuticals, as a filler, and as a viscosity modifier. An example of a major industrial use of starch is in beer production where barley starch is the source of glucose which is fermented by yeast to alcohol.

Starch is a glucan consisting essentially of two polymers of glucose-amylose and amylopectin—which are (α1-4)- and (α1-6)-linked. Specifically, amylose is a linear molecule with D-glucose units linked (α1-4) while amylopectin is a branched structure and has both (α1-4) and (α1-6) linkages. In its natural state in plants, starch exists in granules with crystalline and amorphous areas and is thus described as being “semi-crystalline”. While starch is the major component of such crystals, they also contain some proteins and some lipids. Because of the complex nature of the starch polymer per se, and enormous variation in the physical and chemical properties of starch granules, starch in fact represents a broad range of compounds with there being considerable variation in starches derived from different plants and even between starches derived from different varieties or cultivars of the same plant species.

An important property of starch granules is the temperature at which gelatinization occurs, this being the temperature at which the starch granules begin to lose internal order and crystallinity. There is considerable variation in the gelatinization temperature of starches from different sources. For example, barley starches have a gelatinization temperature of approximately 57° C. while the gelatinization temperature of starches from rice can be greater than 75° C. The gelatinization temperature of starches is important as starch is invariably gelatinized prior to use. For example, food comprising starch such as rice is cooked to aid digestion of the starch and also to provide a more palatable food; barley for beer production is gelatinized in the mashing step so that the starch is amenable to enzymatic degradation to glucose. There are thus many situations where it may be desirable to have available starch with a lower gelatinization temperature so that less energy is required to effect the gelatinization of the starch—barley mashing in beer production for example. A higher gelatinization temperature may be desirable in producing foods with a lower glycemic index.

Starch is synthesised by a pathway comprising a number of enzymes. Regardless of the subsequent synthetic path, glucose is first activated in preparation for starch synthesis by adenosine 5′ diphosphate glucose pyrophosphorylase (AGPase) and the adenosine 5′ diphosphate glucose so produced becomes the substrate for the starch synthases. Granule bound starch synthase (GBSS) is exclusively responsible for the synthesis of amylose and has been extensively studied in rice because different alleles of the gene (the waxy or Wx gene) that codes for GBSS have clear and measurable effects on the appearance and properties of rice grains and rice starch. The amylose content of commercial rice starch varies in the range of 10% to 30% of total starch and it has been found that particular alleles of Wx explains why varieties of rice fall into different amylose classes.

The relative complexity of amylopectin is reflected in the number of enzymes required for its synthesis and has meant that the details of its synthesis are not as well understood as is the case with amylose. Starch synthases (SS) extend the α(1-4) links of amylopectin, starch branching enzymes (SBE) inserts α(1-6) branches in the chains, while starch debranching enzymes (SDE) cleaves the growing chains at α(1-6) linkages in what is believed to be a necessary part of the process of remodeling the growing amylopectin. Each of these enzymes occurs as a number of different isoforms. There are at least three starch synthases (SSI, SSII and SSIII), two starch branching enzymes (SBE A and SBE B) and two forms of SDE (isoamylase and pullulanase).

A link between the gelatinization temperature of rice starch and enzymes of starch bio-synthesis has been made with the finding that a major gene that controls rice gelatinization temperature, as determined by the indirect measure of alkali spreading, genetically maps to a region of chromosome six that is clearly different to the waxy locus (Umemoto et al., 2002). This gene co-segregates with soluble starch synthase IIa (SSIIa) and a gene that affects amylopectin structure (Umemoto et al., 2002) suggesting the cause and affect, or genotype to phenotype, hierarchy of gelatinization temperature is as follows; SSIIa, amylopectin structure, gelatinization temperature. Analysis of near-isogenic lines (NIL) of rice and of the SSIIa gene sequence used to construct the mapping population and NIL (Nipponbare and Kasalsath) provides further support for the hypothesis that SSIIa is the enzyme that is responsible for natural variation in rice starch gelatinization temperature (Umemoto et al., 2004). Western blotting SSIIa in two rice varieties that differed by starch disintegration in both urea and alkali found the amount of SSIIa was reduced in the variety that was more sensitive to both alkali and urea (Jiang et al., 2004).

The data from rice are supported by data derived from peas and barley. Craig and co-workers (1998) found the degree of polymerization of the A chains were much reduced in peas that lack SSII activity in a similar way to that reported by Umemoto and co-workers for rice. It has been shown more recently that mutant barley SSIIa which lacks its catalytic domain due to the occurrence of a premature stop codon in the coding sequence has a higher proportion of chains with a degree of polymerization (DP) 6-11 compared with DP 12-30 in comparison to wild type barley amylopectin (Morell et al., 2003). In addition to this, the starch from the mutant barley had a lower gelatinization temperature, suggesting the amylopectin structure is the primary determinant of starch gelatinization temperature (Morell et al., 2003).

The gelatinization temperature of a starch is also of relevance to the Glycaemic Index (GI) of food comprising the starch. GI is a ranking of foods from 0 to 100 that indicates the extent to which a food will cause a rise in blood sugar levels. The term was first coined in 1981 but has only recently been incorporated into standard dietary practice. The GI allows a comparison of foods gram for gram of carbohydrate. Carbohydrates that break down quickly during digestion have the highest GIs. The blood glucose response is rapid. Carbohydrates that break down slowly, releasing glucose gradually into the blood stream, have low GIs. A low GI is considered to have a numerical value of 55 or less, a moderate GI a value of 56 to 59, and a high GI a value of 70 or more.

It is known that amylopectin structure can have an effect on the GI of food by altering the efficacy of amylopectin breakdown in the gut of animals ingesting the food. However, more important in the present context is the fact that gelatinization temperature is inversely linked to GI: food comprising starch with a low gelatinization temperature has a higher GI than food comprising starch with a high gelatinization temperature.

The GI values of foods must be measured using valid scientific methods. It cannot be guessed by looking at the composition of the food. The GI value of a food is determined by feeding 10 or more healthy people a portion of the food containing 50 grams of digestible (available) carbohydrate and then measuring the effect on their blood glucose levels over the next two hours. For each person, the area under their two-hour blood glucose response (glucose AUC) for this food is then measured. On another occasion, the same 10 people consume an equal-carbohydrate portion of glucose sugar (the reference food) and their two-hour blood glucose response is also measured. A GI value for the test food is then calculated for each person by dividing their glucose AUC for the test food by their glucose AUC for the reference food. The final GI value for the test food is the average GI value for the 10 people.

The GI of a food type, for example rice, can vary significantly within the type. By way of illustration, parboiled Bangladeshi rice has a GI of 32 while boiled Turkish rice has a GI of 139, and boiled white Basmati rice a GI of 58. Similarly, for potatoes, a Kenyan potato has a GI of 24, while a boiled Desiree potato has a GI of 101.

Low-GI foods, by virtue of their slow digestion and absorption, produce gradual rises in blood sugar and insulin levels, and have proven health benefits. Low GI diets have been shown to improve both glucose and lipid levels in people with diabetes (type 1 and type 2). They have benefits for weight control because they help control appetite and delay hunger. Low GI diets also reduce insulin levels and insulin resistance.

Recent studies from Harvard School of Public Health indicate that the risks of diseases such as type 2 diabetes and coronary heart disease are strongly related to the GI of the overall diet. In 1999, the World Health Organisation (WHO) and Food and Agriculture Organisation (FAO) recommended that people in industrialised countries base their diets on low-GI foods in order to prevent the most common diseases of affluence, such as coronary heart disease, diabetes and obesity.

The significance of GIs is becoming more apparent as research is carried out in this field: an intake of low GI foods means a smaller rise in blood glucose levels after meals, which can help people lose weight and can improve the body's sensitivity to insulin, improve diabetes control, and prolong physical endurance. However, there are circumstances when it desirable to have a high GI food available for intake. For example, athletes when participating in an event need an abundant supply of glucose which can be aided by ingestion of food with a high GI.

In view of the health benefits of foods with a low GI but the parallel need for foods with a high GI, there is a need to have available procedures to be able to engineer plants for use as food sources with a desired GI. There is also a need to be able to be able to engineer plants so that the gelatinization temperature of the starch produced by a plant is more suited to its intended use, be that as a food source or in an industrial process.

It is thus an aim of the invention to provide a method of modifying a plant so that starch produced by the plant has an altered amylopectin structure to afford a desired gelatinization temperature. It is furthermore an aim of the invention to provide a plant that has starch with an altered amylopectin structure so that the starch of that plant has a gelatinization temperature that is altered relative to the starch of a parental strain of the plant.

### SUMMARY OF THE INVENTION

According to a first embodiment of the invention, there is provided a process for producing a modified plant comprising starch with an altered amylopectin structure relative to the structure of amylopectin of starch of a parental strain of said plant, the process comprising the steps of:

i) obtaining tissue from a parental plant strain;
ii) changing at least one of the genes encoding enzymes for the synthesis of amylopectin of said tissue so that the amylopectin of starch synthesized by enzymes including the enzyme encoded by the changed gene has an altered structure; and
iii) propagating plants from the tissue prepared in step (ii).

According to a second embodiment of the invention, there is provided a process for producing a modified plant comprising starch with an altered gelatinization temperature relative to the gelatinization temperature of starch of a parental strain of said plant, the process comprising the steps of:

i) obtaining tissue from a parental plant strain;
ii) changing the starch synthase Ia gene of said tissue so that starch synthesized by enzymes including starch synthase Ia encoded by the changed gene has an altered gelatinization temperature; and
iii) propagating plants from the tissue prepared in step (ii).

According to a third embodiment of the invention, there is provided a modified plant comprising starch with an altered gelatinization temperature relative to the gelatinization temperature of starch of a parental strain of said plant.

According to a fourth embodiment of the invention, there is provided a product comprising starch obtained from the modified plant of the third embodiment.

According to a fifth embodiment of the invention, there is provided a process for producing a modified starch synthase IIa gene so that starch synthesized by enzymes including the starch synthase IIa encoded by said modified gene have an altered gelatinization temperature, the process comprising the steps of:

i) identifying variations in starch synthase IIa genes of strains of plants;
ii) correlating a desired gelatinization temperature with specific changes in said genes; and
iii) making said specific changes in the starch synthase Ia gene of a subject plant to obtain said modified gene.

According to a sixth embodiment of the invention, there is provided a modified starch synthase IIa gene product of the process according to the fifth embodiment.

According to a seventh embodiment of the invention, there is provided a modified starch synthase IIa gene, wherein said gene encodes any one or any combination of the following changes relative to the starch synthase IIa gene of a parental plant strain:

in the motif G(LV)RDTV, the last V is replaced by any amino acid residue; and

in the motif (EK)SW(RKE)(AGS)L, the L is replaced by any amino acid residue.

According to an eighth embodiment of the invention, there is provided a method of assessing the gelatinization temperature of the starch of a plant, the method comprising testing for polymorphisms in the starch synthase Ia gene of said plant.

According to a ninth embodiment of the invention, there is provided a process for producing food with a selected Glycaemic Index, the process comprising the steps of:

i) obtaining tissue from a plant to be used as a food source;
ii) changing the starch synthase Ia gene of said tissue so that starch synthesized by enzymes including starch synthase IIa encoded by the changed gene have a gelatinization temperature which yields food with said selected Glycaemic Index;
iii) propagating plants from the tissue prepared in step (ii); and
iv) harvesting food from said plants propagated in step (iii).
According to a tenth embodiment of the invention, there is provided a food product of the process according to the ninth embodiment.

According to an eleventh embodiment of the invention, there is provided a process for producing starch with a selected gelatinization temperature, the process comprising the steps of:

i) obtaining tissue from a plant to be used as a source of starch with said selected gelatinization temperature;
ii) changing the starch synthase Ia gene of said tissue so that starch synthesized by enzymes including starch synthase IIa encoded by the changed gene have said selected gelatinization temperature;
iii) propagating plants from the tissue prepared in step (ii); and
iv) harvesting starch from said plants propagated in step (iii).

According to an twelfth embodiment of the invention, there is provided a starch product of the process according to the eleventh embodiment.

Other aspects of the invention will become apparent from the following detailed description of preferred embodiments thereof. These embodiments will be described with reference to the accompanying drawing, briefly described hereafter.

### BRIEF DESCRIPTION OF THE DRAWINGS

FIG. 1 is a graphical representation of the statistical analysis of polymorphism in rice varieties relative to gelatinization temperature. Specifically, the graph comprising the figure is a one-way Anova 95% confidence interval for each genotypic class.

FIG. 2 is an annotated conceptual translation of the SSIIa gene from the Opus variety of rice. A sequence range of nucleotides 1 to 2,959 is presented.

### DETAILED DESCRIPTION OF PREFERRED EMBODIMENTS

The following abbreviations are used herein:
GI Glycaemic Index
GBSS granule bound starch synthase
kbp(s) kilobase pair(s)
PCR polymerase chain reaction
pfu plaque forming units
SBE starch branching enzyme
SDE starch debranching enzyme
SNP single nucleotide polymorphism
SS starch synthase
SSIIa starch synthase IIa
RT-PCR reverse transcriptase PCR

The term “low GI” is used herein to denote a glucose-containing food, particularly starch, which has a GI of not greater than 55.

The term “high GI” is used herein to denote a glucose-containing food, particularly starch, which has a GI of greater than 70.

The term “altered gelatinization temperature” is used herein to denote any change in temperature that has a beneficial effect on any process utilizing starch that has an altered gelatinization temperature, or has a beneficial effect in the use of the starch per se. In some commercial processes, a lowering of the gelatinization temperature by as little as a half of a degree Celsius effect is of benefit in terms of the cost of a particular process.

The term “parental strain” is used herein to denote any plant from which a modified plant can been prepared by the methods of the invention.

The inventors, through the work to be described below, have identified a link between mutations in the gene encoding SSIIa and the gelatinization temperature of starch formed in plants harbouring the mutant gene. Exploitation of this finding has allowed ways of engineering plants which have starch with a gelatinization temperature that is better suited to the intended use. This engineering applies not only to the SSIIa gene, but to all of the genes involved in the synthesis of amylopectin as a component of starch. In this context, “engineering” includes modification of plant genes that results in synthesis of starch that comprises amylopectin with an altered structure relevant to the amylopectin of plants that have not been engineered.

With reference to the first and second embodiments of the invention as defined above, the parental strain and modified plant derived therefrom can be any starch-producing plant including monocotyledonous or dicotyledonous plants. Typically, the plant is a cereal crop plant. Examples of suitable cereal crop plants include rice, oats, barley, sorghum, maize, wheat, rye, amaranth, rape, and spelt. Other plants amenable to use in the method of the second embodiment include the dicotyledonous plants such as potato and taro. Still further examples of parental plant strains and modified plants derived therefrom are the legumes including alfafa, beans, broom, carob, clover, cowpea, mung bean, mimosa, peas, peanuts, soybeans, tamarind, vetch.

With regard to the steps of the method according to the first and second embodiments, the tissue obtained in step (i) can be any suitable tissue including, but not limited to, seeds, roots, leaves and stems.

In the context of step (ii) of the first and second embodiments, the term “changing” is used in the sense of altering nucleotides of a target gene but also in the sense of inactivating the gene of the parental strain or substantially eliminating the ability of the plant to express a functional protein product of that gene, and inserting a mutated gene into the plant. Methods of carrying out these procedures will be described in the following sections of the specification using the SSIIa gene as an example However, it will be appreciated by one of skill in the art that the procedures are applicable to any one of the genes involved in amylopectin synthesis.

### Methods of Mutating Genes

Nucleotides of the SSIIa gene of the parental strain of a target plant can be altered using any of the methods known in the art. Such alteration comprises the substitution of at least one base pair within the coding region of the gene so that an amino acid residue is changed but a functional protein is still encoded by the changed gene. Specific changes will be illustrated below.

In vitro methods suitable for making a base pair substitution in the SSIIa gene include site-directed mutagenesis (Carter et al. (1985); and Zoller et al. (1982)), cassette mutagenesis (Wells et al. (1985)), restriction selection mutagenesis (Wells et al. (1986)), oligonucleotide-mediated mutagenesis (Adelman et al. (1983)), alanine scanning (Cunningham and Wells (1989)), PCR mutagenesis (Leung et al. (1989)), and saturation mutagenesis (Mayers et al. (1985)).

Chimeric oligonucleotides can also be used to create in vivo site-specific changes in the SSIIa gene: see, for example the procedures described in the papers by Zhu et al. (1999), Zhu et al. (2000), and Kipp et al. (1999) in Gene Targeting Protocols.

As will be discussed below, the SSIIa gene can be mutated by exposing the plant or tissue therefrom to mutagens such as ionising radiation, UV radiation, chemical mutagens, and the like. Mutated plants can then be tested by the methods of the invention for changes in the SSIIa gene, or for production by the modified plant of starch with an altered gelatinization temperature.

### Methods of Gene Inactivation and Replacement

As indicated above, the SSIIa gene can be changed by: (a) inactivating the parental strain gene; or, (b) by substantially eliminating the ability of the plant to express functional protein product of that gene; and then introducing a modified SSIIa gene into the plant from which the desired protein is expressed. To first deal with (a), the SSIIa gene can be mutated by any method which results in the expression of a non-functional protein product of the gene. It will be understood by those skilled in the art that in some cases, a protein may still be expressed, but the expressed protein will not be functional. For example, when the mutation is a mutation which results in formation of a stop codon, a truncated protein that is not a functional protein can be produced.

The SSIIa gene can be mutated by inserting at least one additional base pair into the gene. The insertion may create a frame shift which results in expression of a truncated non-functional protein, or no protein expression. The insertion can comprise translation and/or transcription stop signals. The insertion can be a single base pair, or a plurality of base pairs. For example, the insertion can be a gene which encodes a selectable marker. As used herein, “selectable marker” refers to a gene or nucleic acid sequence encoding a trait or phenotype which can be selected or screened for in an organism. Examples of selectable markers include antibiotic resistance genes, carbon source utilisation genes, amino acid production genes and the like. Selectable markers for use in plants are well known in the art and are described in, for example, Ziemienowizc A. (2001). Methods for mutating genes in plants by introducing insertions into genes of the plant are described in, for example, Krysan et al. (2002); Greco et al. (2001); Gelvin (2000); Henikoff and Comai (2003).

An insertion can be made in a gene using, for example, transposon mutagenesis, homologous recombination or site specific recombination. An example of site-specific recombination is the cre-lox recombination system of bacteriophage P1 (see Abremski et al. (1983); Sternberg et al. (1981)), which has been used to promote recombination of specific locations on the genome of plant cells (see, for example, U.S. Pat. No. 5,658,772). A further example of site-specific recombination is the FLP recombinase system of Saccharomyces cerevisiae (see, for example, U.S. Pat. No. 5,654,182). Activity of the FLP recombinase system has been demonstrated in plants (see Lyznik et al. (1996); Luo et al. (2000)).

The gene encoding the functional protein can be disrupted by introducing an insertion by homologous recombination as described in, for example, U.S. Pat. No. 6,750,379.

The SSIIa gene can be changed by transposon mutagenesis. Transposons, retrotransposons and methods for the mutagenesis of genes using transposons and retrotransposons in plants are described in, for example, Belmetzen (1996); Voytas (1996); Hiroshik et al. (1996); and, U.S. Pat. No. 6,720,479.

The SSIIa gene can also be changed by deleting at least one base pair from the gene that results in a reduction or elimination of expression of the functional protein from that gene. The deletion can be any size, and in any location in the gene encoding the SSIIa protein, provided the deletion results in elimination of expression of a functional protein. The deletion can be in the coding sequence or the 5′ non-coding region, such as the promoter, which prevents production of a transcript. The deletion can alternatively be in an intron or at an intron/exon boundary. The deletion can furthermore be in the 3′ non-coding region. The deletion can comprise a substantial portion of the gene, or the entire gene.

Methods for the production of deletion mutations in plants are described, for example, in Li (2001) and Henikoff and Comai (2003).

The SSIIa gene can alternatively be changed by the substitution of at least one base pair within the gene so that a functional protein is no longer encoded. The substitution can be in the coding or non-coding portion of the gene. The substitution can be such that the formation of a stop codon (TGA, TAG, TAA) results, or an amino acid substitution that results in expression of a non-functional protein. The substitution can be in a non-coding portion of the gene which results in reduction or elimination in production of RNA transcript. The substitution can be introduced into the gene using any of the methods known in the art.

The SSIIa gene can be mutated by, for example, exposing the plant or tissue therefrom to mutagens such as ionising radiation, UV radiation, chemical mutagens, and the like. Examples of ionising radiation include beta, gamma or X-ray radiation. Examples of chemical mutagens include ethyl methyl sulfonate, methyl N-nitrosoguanidine, N-nitroso-N-ethylurea, N-nitroso-N-methylurea, ethidium bromide, and diepoxybutane. The time and dosage for exposure of the plant or tissue to the mutagen will vary depending on the organism and the mutagen that is used, and can be readily determined by the person skilled in the art.

Mutation of the SSIIa gene can be effected using recombinant DNA technology to delete, insert or alter the sequence of the gene. For example, the gene can be mutated by inserting a nucleic acid sequence into the gene such that the gene is no longer capable of expressing a functional protein. The nucleic acid sequence can be any nucleic acid sequence that disrupts expression of the gene. For example, the nucleic acid sequence that is inserted can be a selectable marker. Methods for inserting nucleic acid molecules into the genes of plants are described in, for example, in Hiatt (1993).

Mutants generated by any of the above methods, or naturally occurring mutants, can be screened for by any methods known in the art. For example, mutants can be identified using TILLING (Target Induced Local Lesion in Genomes). Typically, in TILLING the SSIIa gene of a plant to be screened is amplified and annealed with the amplified SSIIa gene of the parental strain, and heteroduplexes are detected to determine whether the first-mentioned SSIIa gene has been mutated. Methods for TILLING have been described, for example, by McCallum et al. (2000). Typically, TILLING is carried out following mutagenesis. However, it will be appreciated by those skilled in the art that TILLING can also be employed to identify plants with naturally occurring mutations in the SSIIa gene.

With regard to (b) above, the ability of the plant to express functional protein product of the SSIIa gene can be effected by any of the methods known to those of skill in the art. For example, the amount of RNA transcribed from the parental strain SSIIa gene can be eliminated. Also, the ability of the plant to translate protein from the RNA transcripts of the parental strain SSIIa gene can be eliminated.

The nucleic acid molecule which eliminates expression of a functional protein product of the parental strain SSIIa gene can be an antisense molecule. As used herein, an “anti-sense molecule” is a nucleic acid molecule comprising a sequence that is complementary to a specific DNA or RNA target sequence and is capable of hybridising to the target sequence so as to eliminate transcription or translation of the target sequence. The term “hybridise” will be understood by those skilled in the art to refer to a process by which a nucleic acid strand anneals with a substantially complementary strand through base pairing. Examples of anti-sense molecules include: anti-sense nucleic acid, including single stranded or double stranded anti-sense DNA or RNA, co-suppressor DNA or RNA, interference RNA (including RNAi, siRNA, hpRNA, ihpRNA), ribozymes. The anti-sense molecule may be an anti-sense RNA. As used herein, an anti-sense RNA refers to an RNA molecule that is complementary to, or at least partially complementary to, and therefore capable of forming a duplex with, a target RNA molecule to thereby eliminate translation from the target RNA molecule. The anti-sense RNA molecule can be complementary, or partially complementary, to a coding or non-coding region of the target RNA molecule. The anti-sense RNA molecule can be any length which reduces or eliminates expression of the functional protein. Methods for the use of anti-sense RNA for eliminating expression of a gene are known and are described in, for example, U.S. Pat. No. 5,107,065; Smith et al. (1988); Van der Krol et al. (1988); Rothstein et al. (1987); Bird et al. (1991); Bartley et al. (1992); Gray et al. (1992). The anti-sense molecule can be interference RNA (including RNAi, siRNA, hpRNA and ihpRNA). As used herein, interference RNA refers to dsRNA-mediated interference of gene expression in which double stranded RNA that is complementary to a target nucleic acid sequence is used to selectively reduce or eliminate expression of the target gene. Methods for the production and use of RNAi are known in the art and are described in, for example, C. P. Hunter (1999); Hamilton et al. (1999); Ding (2000).

The anti-sense molecule can be a ribozyme. As used herein, the term “ribozyme” refers to an RNA molecule comprising sequence complementary to a target RNA sequence when the complementary sequence hybridises with the target sequence. Methods for the production and use of ribozymes for reducing or eliminating expression of genes are known and described in, for example, Kim and Cech, (1987); Reinhold-Hurek and Shub (1992); U.S. Pat. No. 5,254,678; Methods in Molecular Biology (1997).

The nucleic acid molecule which eliminates expression of a functional protein product of the parental strain SSIIa gene can be a co-suppressor RNA molecule. A co-suppressor RNA molecule is homologous to at least a portion of the RNA transcript of the gene to be suppressed. Methods for reducing or eliminating gene expression using co-suppressor RNA are known and are described in, for example, U.S. Pat. No. 5,231,020; Krol et al. (1988); Mol et al. (1990); Grierson et al. (1991); Krol et al (1990); Napoli et al. (1990); U.S. Pat. No. 5,231,020; WO95/34668; Angell and Baulcombe (1997).

Typically, a nucleic acid molecule which reduces or eliminates expression of the function protein is an oligonucleotide, suitably an anti-sense oligonucleotide. Antisense oligonucleotides can be any length that is sufficient to reduce or eliminate expression of the SSIIa gene. Suitably, the anti-sense oligonucleotides are greater than 10 bp in length. More suitably, the anti-sense oligonucleotides are between 10 and 100 bp in length, more typically between 12 and 50 bp in length. The anti-sense oligonucleotides can be any of the abovementioned antisense molecules. The oligonucleotides can be synthesised manually or by an automated synthesiser using methods known in the art.

The nucleic acid molecule which eliminates expression of a functional protein product of the parental strain SSIIa gene can be part of a vector. Typically, the vector is an expression vector. As used herein, an “expression vector” refers to a nucleic acid construct in which a nucleic acid molecule which reduces or eliminates expression of the functional protein is operably linked to a vector whereby the vector sequence specifies expression of nucleic acid molecules from the expression vector when the vector is introduced into the plant. Typically, the nucleic acid molecules are anti-sense molecules or co-suppressor molecules. Suitable vectors for the expression of nucleic acid molecules in organisms are known and include any vectors that are suitable for expression of RNA in a plant. For example, Ti and Ri plasmid derived vectors for use with Agrobacterium tumefaciens are suitable vectors for plants. Suitable Ti and Ri plasmid derived vectors include those disclosed in U.S. Pat. No. 4,440,838; Weissbach and Weissbach (1988); Geierson and Corey (1988); Miki and Iyer (1997); Barton and Chilton (1983).

Replication deficient viral vectors can be employed for expression of RNA in a plant. Such vectors include, for example, wheat dwarf virus (WDV) shuttle vectors such as aspW1-I1 and PW1-GUS (see Ugalci et al. (1991)).

The anti-sense molecule or vector can be introduced into the cells by any methods known in the art, such as those described in, for example, Hannon (2002); Bernstein et al (2002); Hutvagner et al. (2002); Brummelkamp (2002). Methods for introduction of anti-sense molecules and constructs into plants include transfection, transformation, electroporation, Agrobacterium tumefaciens-mediated transformation, microprojectile-mediated transformation (see, for example, Glick and Thompson (1993); Sambrook et al. (1989); Duan et al. (1996).

Having mutated a parental strain SSIIa gene, or substantially eliminated the ability of the plant to express functional protein product of that gene, an exogenous gene that encodes the desired SSIIa protein can be introduced into the plant by any of the methods known to those of skill in the art. Introduction to the plant is typically by way of a DNA construct which includes the mutated gene. The term “construct” includes vectors such as plasmids, cosmids, viruses, and the like as well as naked DNA per se. Control elements which can be included in constructs will be known to those of skill in the art. Examples of such elements are promoters, enhancers, polyadenylation signals and transcription terminators.

A binary vector system can be used to introduce the exogenous SSIIa gene into a plant. In this method, a gene cassette comprising the modified SSIIa can be ligated into binary vectors carrying: i) left and right border sequences that flank the T-DNA of the Agrobacterium tumefaciens Ti plasmid; ii) a suitable selectable marker gene for the selection of transformed cells or plants; iii) origins of replication that function in A. tumefaciens or Escherichia coli; and, iv) antibiotic resistance genes that allow selection of plasmid transformed cells of E. coli and A. tumefaciens. The binary vectors can then be introduced either by electroporation or tri-parental mating into A. tumefaciens strains carrying disarmed Ti plasmids such as strains LBA4404, GV3101 and AGL1 or into A. rhizogenes strains such as R4 and NCCP1885. These Agrobacterium strains can be co-cultivated with suitable plant explants or intact plant tissue and the transformed plant cells and/or regenerant shoots selected using an agent that allows the presence of the selectable marker gene to be determined. Suitable selectable marker genes can be used to confer resistance to antibiotics or herbicides or to produce a molecule that can be assayed fluorometrically or chemically.

Direct insertion can also be used to introducing an exogenous SSIIa gene into a plant. A gene cassette comprising the exogenous gene can be micro-injected into isolated plant cells which are then selected for introgression of the gene into the genome. Alternatively, the gene cassette can be co-precipitated onto gold or tungsten particles along with a plasmid encoding a chimeric selectable marker gene. The encoated particles or projectiles are accelerated into plant cells or tissues.

With regard to methods of transforming plant cells and of regenerating plants, there may be mentioned in particular the following patents and patent applications: U.S. Pat. No. 4,459,355; U.S. Pat. No. 4,536,475; U.S. Pat. No. 5,464,763; U.S. Pat. No. 5,177,010; U.S. Pat. No. 5,187,073; EP 267,159; EP 0 604 662; EP 0 672 752; U.S. Pat. No. 4,945,050; U.S. Pat. No. 5,036,006; U.S. Pat. No. 5,100,792; U.S. Pat. No. 5,371,014; U.S. Pat. No. 5,478,744; U.S. Pat. No. 5,179,022; U.S. Pat. No. 5,565,346; U.S. Pat. No. 5,484,956; U.S. Pat. No. 5,508,468; U.S. Pat. No. 5,538,877; U.S. Pat. No. 5,554,798; U.S. Pat. No. 5,489,520; U.S. Pat. No. 5,510,318; U.S. Pat. No. 5,204,253; U.S. Pat. No. 5,405,765; EP 0 442 174; EP 0 486 233; EP 0 486 234; EP 0 539 563; EP 0 674 725; and the international applications having the publication numbers WO 91/02701 and WO 95/06128.

Regenerant plants can be selected for presence of the marker gene and production of starch with the altered gelatinization temperature.

With further reference to step (ii) of the second embodiment method, mutations of the SSIIa gene which result in starch with a lowered gelatinization temperature include the following:

in the motif G(LV)RDTV, the V is replaced by M; and

in the motif (EK)SW(RKE)(AGS)L, the L is replaced by F.

Turning to the third step of the method of the first and second embodiments, propagation of the plant tissue from step (ii) can be by any of the methods known to those of skill in the art. For example, plants can be propagated by any of the methods described in George (1993).

The modified plant of the third embodiment can be any starch-producing plant. Examples of such plants are given above in the description of the second embodiment.

The plant product of the fourth embodiment can be starch per se or material such as rice, pasta, bread, noodles, and potato.

With regard to the fifth embodiment of the invention, the identification of variations in the SSIIa gene can be any of the gene analysis techniques known to those of skill in the art including TILLING (see above). Other suitable techniques include enzymatic approaches (restriction enzymes type II, Cleavase and Resolvase, DNA polymerase, and ligase), single- and double-stranded conformation assays, heteroduplex analysis, and DNA sequencing, high-density oligonucleotide arrays for hybridization analysis, minisequencing primer extension analysis, fiberoptic DNA sensor array, denaturing high-performance liquid chromatography (DHPLC), mass spectrometry (mass and charge) or fluorescence exchange-based techniques Kristensen et al (2001). On identifying variations, simply determining the gelatinization temperature of starch produced by a plant in which variations have been found allows establishing a correlation between a particular variation and gelatinization temperature (step (ii) of the method).

Changing the SSIIa gene in step (iii) of the fifth embodiment method can be done by any method by which nucleotides can be altered including the methods referred to above in the description of step (ii) of the second embodiment.

A modified SSIIa gene produced by the process of the fifth embodiment—such a product being the sixth embodiment of the invention—has utility in the production of plants in accordance with the second embodiment method.

The seventh embodiment will be described in greater detail below in the examples of the invention.

As a consequence of being able to correlate gelatinization temperature with variations in the SSIIa gene (see the fifth embodiment above), the correlation data allows assessing the gelatinization temperature of starch produced by a particular plant merely by testing for SNPs in the plant (the eighth embodiment defined above).

The testing can be done by any of the methods known to those of skill in the art including those referred to above.

With regard to the ninth and eleventh embodiments, steps (i) to (iii) of each method are carried out in essentially the same way as steps (i) to (iii) of the second embodiment method. Step (iv) of the ninth and eleventh embodiment methods can be carried out by any suitable procedure.

With reference to step (iv) of the eleventh embodiment method, harvesting starch from a modified plant can be carried out by any of the procedures known to those of skill in the art.

The food product of the tenth embodiment includes cereals such as rice and corn, potatoes, and wheat.

The starch product of the twelfth embodiment includes rice, pasta, bread, noodles, and potato.

Particular embodiments of the invention will now be illustrated with reference to the following non-limiting examples.

### Example 1

### Determination of Gelatinization Temperatures in Rice

In this example, we illustrate the identification of mutations in the rice SSIIa gene which impact of the gelatinization temperature of starch produced by enzymes including the mutated SSIIa.

### Materials and Methods

### Gelatinization Temperature Determination

Rice starch peak temperature of gelatinisation was measured by differential scanning calorimetry.

### Bioinformatics and Statistical Analysis

Blastn and blastx were used to identify sequences with homology to C73554 within the National Center for Biotechnology Information (NCBI; http://www.ncbi.nlm.nih.gov/) and Gramene (http://www.gramene.org/) data bases. Sequence aliginent was undertaken using ClustalW within MacVector™ 6.5.1 (Oxford Molecular Group). Primers were designed using MacVector™ 6.5.1. Statistical analysis, including test of homogeneity of variance, 1-way Anova and Tukey HSD, was undertaken using the software package SPSS.

### DNA Extraction, PCR, Sequence Analysis and Genotyping

Genomic DNA was extracted using a Qiagen Dneasy® 96 Plant Kit (Qiagen GMbH, Germany). DNA preparations were diluted with TE buffer to a final concentration of approximately 10 ng per μl. Oligonucleotide primers were synthesised by Proligo Australia Pty Ltd. PCR was performed using a Perkin Elmer, Gene Amp PCR system 9700. The reaction volume was 25 μl containing 20 ng of extracted genomic DNA, 2.5 mM MgCl₂, 200 μM total dNTPs, 1 unit of Platinum® Taq DNA Polymerase (Gibco BRL®), 1× Gibco® PCR Buffer (minus MgCl₂) and 0.2 μM of each forward and reverse primer. Cycling conditions were 94° C. for 2 minutes followed by 30 cycles of 94° C. for 30 s, 55° C. for 30 s and 72° C. for 1 minute followed by a final extension of 72° C. for 7 minutes.

Prior to sequencing, PCR products were purified using a montage PCR filter device, Millipore Corporation. Sequence reactions was performed on PCR products in with both forward and reverse PCR primers using BigDye Terminator version 3.1, Applied Biosystems, and the completed reactions purified by ethanol precipitation. The reaction products were analysed on an Applied Biosystems 3730 Genetic Analyser.

### Results

### Polymorphism Identification and Assay

The DNA sequence of cDNA clone C73554 which was sequenced and genetically mapped by Umemoto et al. (2002) was used as the query sequence in a Blastn search for similar rice sequences. Blastn identified several database entries with DNA sequence similar to C73554. Alignment of these sequences with each other and comparison of the conceptual translations with the translated sequence in Umemoto et al. (2002) determined the cDNA AF419099 (NCBI accession) is a full length version of C73554. The first base pair of C73554 aligned to base pair 1239 of AF419099. A Blastn search of the Gramene database using both AF419099 and C73554 as the query sequence found BAC clone AP003509 contains the gene from which AF419099 and C73554 is transcribed. ClustalW alignment of AP003509 and AF419099 confirmed the gene consists of 8 exons. Each of the 8 exons were amplified in rice varieties Opus, Doongara, and Langi and the sequence aligned and compared using ClustalW. The gelatintisation temperature of Opus, Doongara, and Langi are 69° C., 74° C. and 78° C. respectively. A single SNP(SNP1) was identified, a “G” to “A” transition in exon 8 corresponding to base pair 2412 of AF419099. This nucleotide change results in a conservative amino acid change from methionine (ATG) to valine (GTG). Langi and Doongara carry the “G” allele while Opus and Nipponbare carry the “A” allele. Nipponbare has a starch gelatinization temperature of 68° C. This region of exon 8 was analysed in a further 77 varieties that differed by gelatinization temperature. Another polymorphism was identified which led to an amino acid change. In this case two adjacent bases that correspond to base pairs 2543 and 2544 of AF41099 were found to be either “GC” (leucine, CTC) or “TT” (phenylalanine, TTC). Although this polymorphism differs by two bases, it is called SNP 2 for the sake of convenience and because only the second base change affects the amino acid sequence.

Umemoto and co-workers (2004) identified a total of three SNPs which resulted in amino acid changes when comparing Nipponbare and Kalsath SSIIa DNA which they called SNP1, SNP2 and SNP3. Umemoto SNP1 was deemed not to affect enzyme properties by virtue of its location near the amino terminal (Umemoto et al., 2004) and was not included in the analysis undertaken here. Umemoto SNP2 (SNP3 for this analysis) corresponds to base pair 2013 of AF41099. Umemoto SNP2 and SNP3 are called SNP3 and SNP1 respectively in this analysis. Umemoto and co-workers did not identify SNP2 identified in this work and consequently could not clearly delineate starch gelatinisation temperature classes. Analysis of these three SNPs in all 77 different genotypes found that only four of the eight possible combinations were represented (Tables 1, 2, 3, 4 and 5).

### Gelatinization Temperature Determination and Statistical Analysis

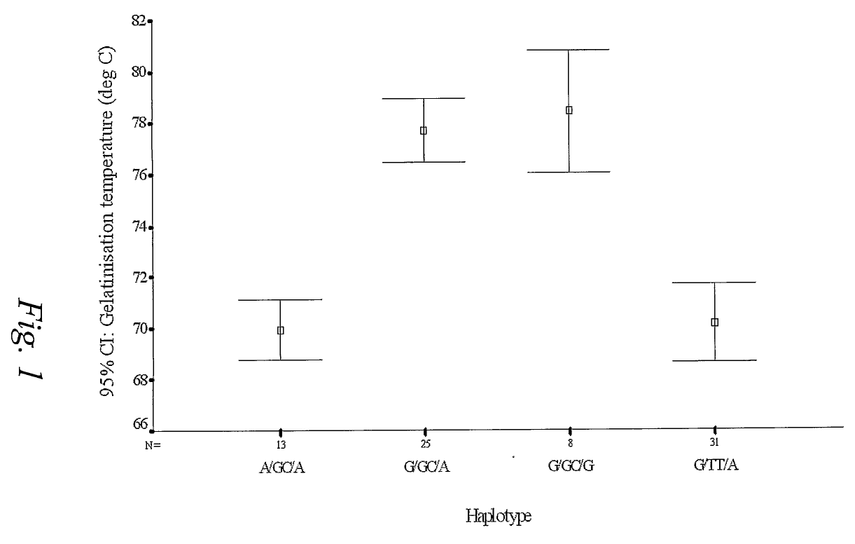

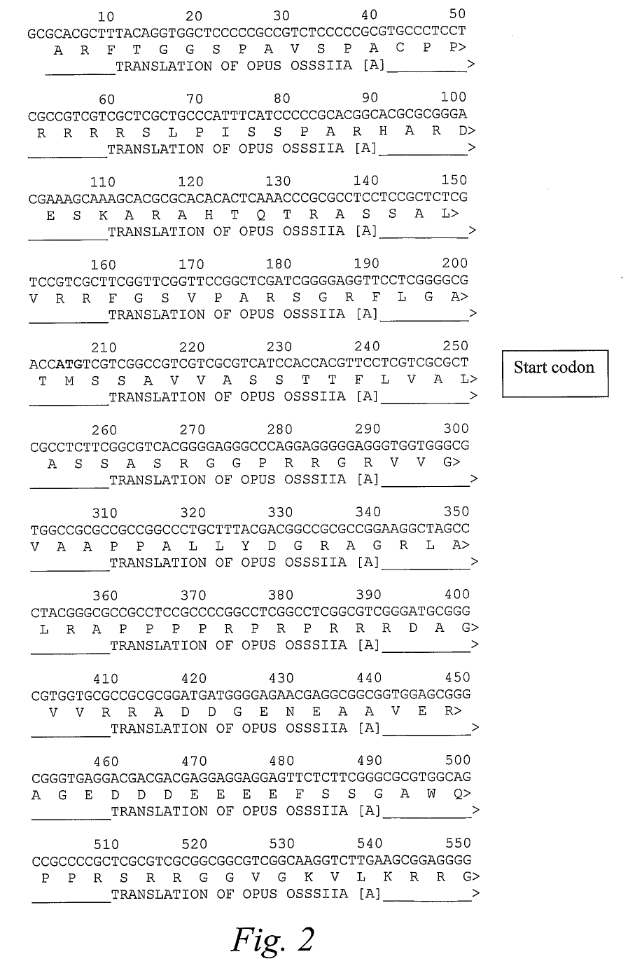

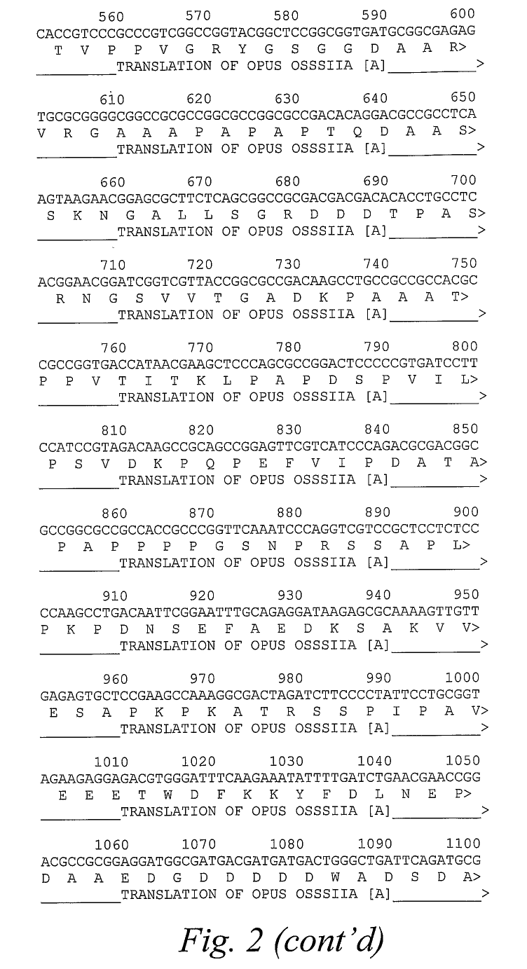

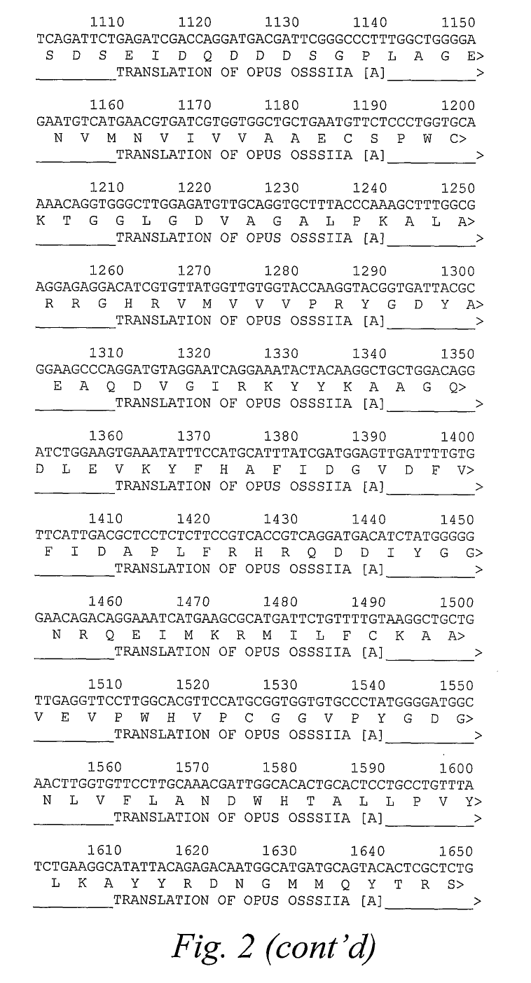

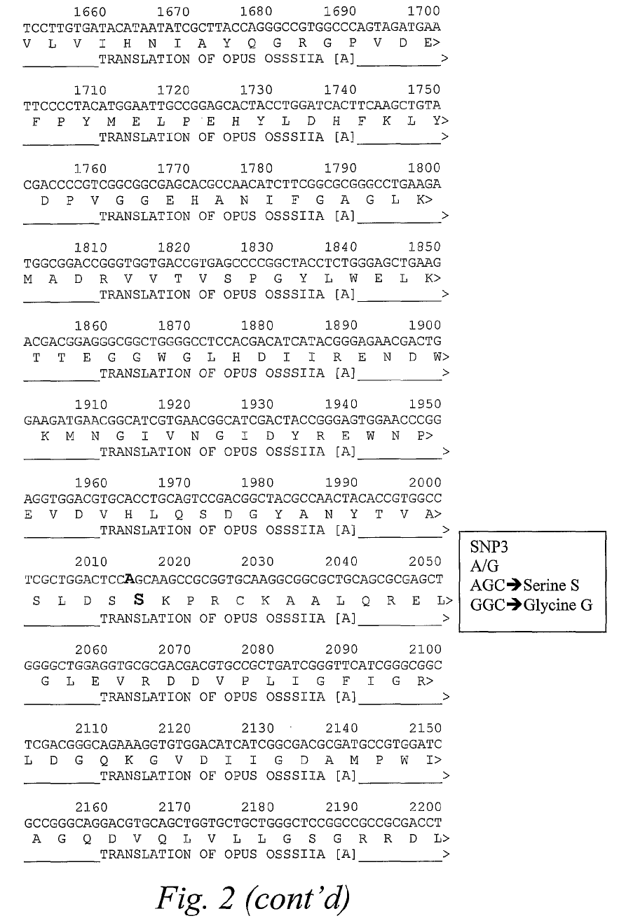

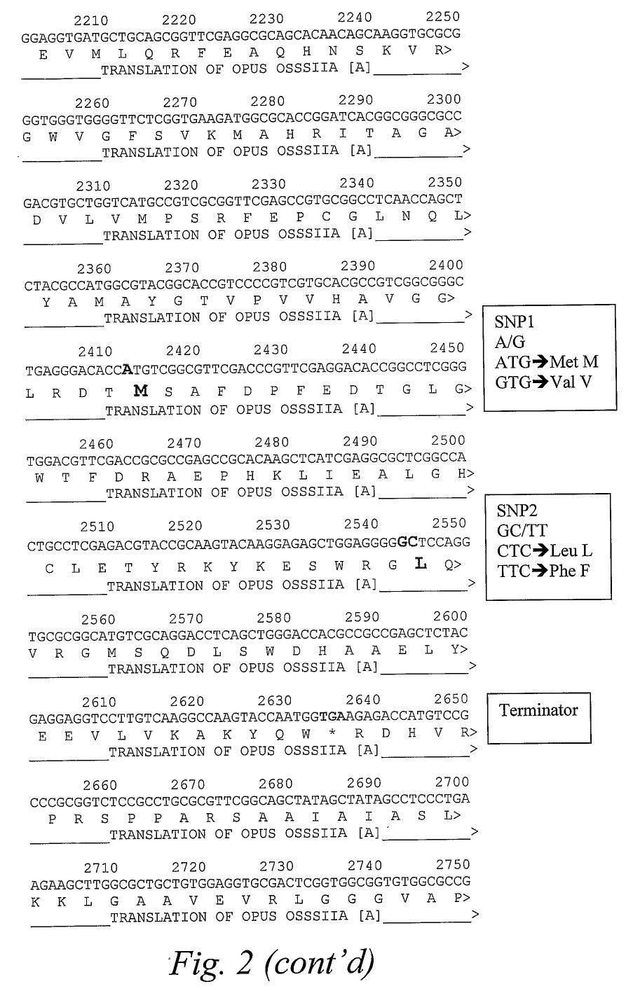

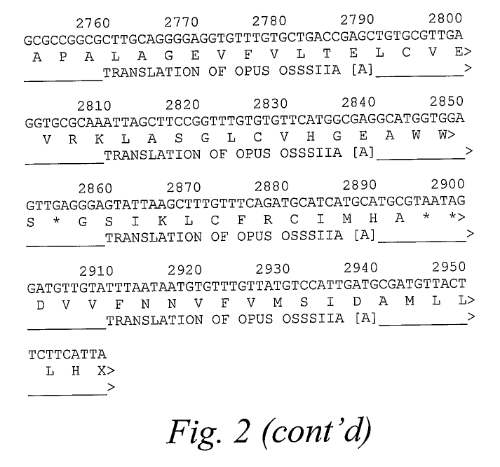

When grouped by genotypic class (test of statistical significance; no significant difference in variance, 1-way Anova, Tukey HSD Post Hoc Test), two gelatinization temperature classes are evident (Table 1 and FIG. 1). The low gelatinization class is composed of the genotypic classes A/GC/A and G/TT/A while the high gelatinization temperature class is composed of the genotypic classes G/GC/A and G/GC/G. An annotated conceptual translation of the SSIIa gene of the variety Opus is presented in FIG. 2 to illustrate the overall position of polymorphisms in the gene (see also SEQ ID NO: 1).

TABLE 1
Summary of SNPs

| | | | | | 95% |
|---|---|---|---|---|---|
| | | | | | confidence |
| | No. of | Mean gelat- | Stand- | | interval for |
| | genotypes | inization | ard | | mean lower |
| | with | temperature | devi- | Std | bound/upper |
| Haplotype | haplotype | (° C.) | ation | error | bound |
| 1: A/GC/A | 13 | 69.9 | 2.0 | 0.54 | 68.8/71.1 |
| 2: G/GC/A | 25 | 77.7 | 2.9 | 0.59 | 76.4/78.9 |
| 3: G/GC/G | 8 | 78.4 | 2.8 | 1.00 | 76.0/80.8 |
| 4: G/TT/A | 31 | 69.9 | 4.2 | 0.73 | 68.4/71.4 |
| Total | 77 | 73.2 | 5.18 | 0.60 | 72.5/74.9 |

TABLE 2
Cultivars of haplotype 1 (A/GC/A)

| | Gelatinization | Exon 8 | Exon 8 | Exon 8 |
|---|---|---|---|---|
| Cultivar | Temperature | SNP 1 | SNP 2 | SNP 3 |
| Millin | 68 | A | GC | A |
| Nipponbare | 68 | A | GC | A |
| Ardito | 68 | A | GC | A |
| Ari Combo | 68 | A | GC | A |
| Opus | 69 | A | GC | A |
| Haenukai | 69 | A | GC | A |
| Jarrah | 69 | A | GC | A |
| Akitakomachi | 71 | A | GC | A |
| Matsuribare | 71 | A | GC | A |
| Opus/Matsuribane | 71 | A | GC | A |
| Ari Combo/Vialone | 71 | A | GC | A |
| Nano | | | | |
| Koshihikari | 73 | A | GC | A |
| Somewake | 74 | A | GC | A |

TABLE 3
Cultivars of haplotype 2 (G/GC/A)

| | Gelatinization | Exon 8 | Exon 8 | Exon 8 |
|---|---|---|---|---|
| Cultivar | Temperature | SNP 1 | SNP 2 | SNP 3 |
| Langi/Inga/Pelde | 69 | G | GC | A |
| Dellmont | 74 | G | GC | A |
| Doongara | 74 | G | GC | A |
| Dawn | 75 | G | GC | A |
| Domsiah I | 75 | G | GC | A |
| Della | 76 | G | GC | A |
| YRF207/L202 | 76 | G | GC | A |
| Bluebelle | 76 | G | GC | A |
| Moroberekan | 77 | G | GC | A |
| YRF203 | 78 | G | GC | A |
| Langi | 78 | G | GC | A |
| L203/71048-20C′i/ | 78 | G | GC | A |
| Hungarian No. 1/ | | | | |
| Rexmont | | | | |
| Gulfmont | 78 | G | GC | A |
| Milagrossa | 79 | G | GC | A |
| YRL102 | 79 | G | GC | A |
| Goolarah | 79 | G | GC | A |
| Bluebelle/M9/Pelde/ | 79 | G | GC | A |
| YRL304/YRF207 | | | | |
| Hungarian No 1 | 79 | G | GC | A |
| Rexmont | 79 | G | GC | A |
| Pelde | 80 | G | GC | A |
| L202 | 80 | G | GC | A |
| YRL118/Inga | 81 | G | GC | A |
| YRL118 | 81 | G | GC | A |
| YRL118/Inga/M9/2 | 82 | G | GC | A |
| 13d.25 | | | | |
| M201/YRM3/Bogan/ | 82 | G | GC | A |
| h989-4s | | | | |

TABLE 4
Cultivars of haplotype 3 (G/GC/G)

| | Gelatinisation | Exon 8 | Exon 8 | Exon 8 |
|---|---|---|---|---|
| Cultivar | Temperature | SNP 1 | SNP 2 | SNP 3 |
| Dumsorkh | 73 | G | GC | G |
| Moosa Tarom | 76 | G | GC | G |
| Domsiah II | 78 | G | GC | G |
| Amber | 79 | G | GC | G |
| Teqing | 79 | G | GC | G |
| RIL266 | 80 | G | GC | G |
| Basmati 370 | 80 | G | GC | G |
| IRBB60 | 83 | G | GC | G |

TABLE 5
Cultivars of haplotype 4 (G/TT/A)

| | Gelatinisation | Exon 8 | Exon 8 | Exon 8 |
|---|---|---|---|---|
| Cultivar | Temperature | SNP 1 | SNP 2 | SNP 3 |
| Vialone Nano | 62 | G | TT | A |
| Calrose | 63 | G | TT | A |
| Shimuzi mochi | 65 | G | TT | A |
| YRM54 | 66 | G | TT | A |
| Amaroo | 67 | G | TT | A |
| Illabong | 67 | G | TT | A |
| YRM62 | 67 | G | TT | A |
| Haenukai/Illabong | 67 | G | TT | A |
| M101 | 68 | G | TT | A |
| Wakamizu | 68 | G | TT | A |
| M401 | 68 | G | TT | A |
| M9 | 69 | G | TT | A |
| YRM42 | 69 | G | TT | A |
| YRM42/Wakamizu | 69 | G | TT | A |
| YRL118/Rexmont | 69 | G | TT | A |
| Bogan | 70 | G | TT | A |
| Kairyo Mochi | 70 | G | TT | A |
| M202/YRL96/Ardito/ | 70 | G | TT | A |
| YRM54 | | | | |
| YRM54/Akitakomachi | 70 | G | TT | A |
| M103 | 71 | G | TT | A |
| Calmochi 202 | 71 | G | TT | A |
| M202 | 72 | G | TT | A |
| Echuca | 72 | G | TT | A |
| YRM54/Rexmont | 72 | G | TT | A |
| YRL101 | 73 | G | TT | A |
| YRW4 | 73 | G | TT | A |
| IRBB59 | 73 | G | TT | A |
| YRM54/Wakamizu | 73 | G | TT | A |
| YRM62/M103/M201/ | 79 | G | TT | A |
| Calrose | | | | |
| YRM2/M101/M103/ | 80 | G | TT | A |
| Koshihikari | | | | |
| YRL118/Rexmont | 80 | G | TT | A |

### Discussion

Using 77 different varieties of rice that differ by gelatinization temperature, targeted sequence analysis of a portion exon 8 of the SSIIa in rice found three polymorphisms that result in a change in amino acid sequence and of these three, two that explain starch gelatinization temperature differences. Four of the possible eight combinations of these polymorphisms were found in these varieties, namely AIGCIA, G/GC/A, G/GC/G and G/TT/A. The first group were those with an average starch gelatinization temperature of 70° C. which was composed of 13 varieties with genotype A/GC/A and 33 varieties with genotype G/TT/A. The second group had an average starch gelatinization temperature of 78° C. and this group was composed of the genotypes G/GC/A (13 varieties) and G/GC/G (31 varieties).

SNP 3 does not appear to affect gelatinization temperature. The high starch gelatinization temperature class has the combination of “G” at SNP1 (valine) and “GC” at SNP2 (leucine) while the low starch gelatinization temperature class is either A/GC or G/TT. This suggests changing either amino acid, valine or leucine, affects SSIIa enzyme activity such that amlypectin structure is affected which in turn results in a lower gelatinization temperature.

Umemoto and co-workers (2004) identified the polymorphisms that are named SNP1 and SNP3 in this work and concluded that both had to change from “G” to “A” to make SSIIa defective and lower gelatinization temperature. The data here does not support this assertion since haplotype 4 (Table 1) has a low starch gelatinization temperature yet is has a G at one of the SNP positions. The data of Umemoto and co-workers (2004) did not allow determination of the relative importance of each SNP and suggested there may be another unidentified SNP which also affects SSIIa activity. The SNP to which they refer may be SNP2 in this analysis.

### Example 2

Identification of SNPs in Other Plant Species

On the basis of the SNPs identified in the rice SSIIa gene, we conducted experiments to determine whether the same mutations occur in other plant species.

### Materials and Methods

The amino acid sequence of rice SSIIa was used as the query sequence in a blastp search of the National Center for Biotechnology Information (NCBI) database (http://www.ncbi.nlm.nih.gov/aa) using the NCBI default parameters. The protein sequence corresponding to rice SSIIa orthologs from commercial monocot species was retrieved from the NCBI database and aligned using ClustalW available at the European Bioinformatics Institute (EMBL-EBI) (http://www.ebi.ac.uk/embl/).

### Results

In Table 6 below, there is presented codons for each of the equivalent SNP positions identified in rice, wheat, barley and maize.

TABLE 6
Codons at positions equivalent to rice SNPs

| | SNP1 | SNP2 | SNP3 |
|---|---|---|---|
| Species | (AA/Codon) | (AA/Codon) | (AA/Codon) |
| Wheat | Valine/GTG | Leucine/CTC | Glycine/GGC |
| Barley | Valine/GTG | Leucine/CTC | Glycine/GGC |
| Maize | Valine/GTG | Leucine/CTC | Glycine/GGA |
| High gel | Valine/GTG | Leucine/CTC | AGC/Serine or |
| temp rice | | | GGC/Glycine |
| Low gel | Methionine/ATG | Leucine/CTC | AGC/Serine |
| temp rice | | | |
| Low gel | Valine/GTG | TTC/Phenylalanine | AGC/Serine |
| temp rice | | | |

The pattern is identical for all species (other than rice) and is the same as that found in rice varieties that have a high gelatinization temperature, indicating that the SNPs identified in rice can be used in other species to obtain the desired gelatinization temperature.

### Example 3

### Modification of Rice

In this example, we demonstrate how the SSIIa gene of a rice variety can be changed so that starch produced by the modified species has an altered gelatinization temperature.

### Materials and Methods

The three basic steps of the process are as follows:
Step 1: Knockout of endogenous SSIIa activity in the target rice variety of high starch gelatinization temperature.
Step 2: Isolation of a rice SSIIa cDNA clone from a rice variety with low starch gelatinization temperature and production of a suitable plant protein expression construct.
Step 3: Transformation of the target rice variety produced in step 1 with the DNA construct produced in step 2.

### Knockout of Endogenous SSIIa Activity in the Target Rice Variety

The endogenous SSIIa gene of the rice variety Basmati 370 (starch gelatinization temperature 80° C.) was disabled according to the protocol of Li et al. (2001). Wild type Basmati 370 seeds were subjected to fast neutron bombardment in the range of 20-30 Gy. The mutagenized seeds were planted into the field and the seeds from individual plants harvested at maturity. DNA was isolated from 50 000 M₂ plants using Qiagen Dneasy® 96 Plant Kit (Qiagen GMbH, Germany) and placed in 25 pools of 2000 individuals. PCR was employed to screen for SSIIa deletion mutants using primers external to the SSIIa gene which amplified a ˜6 kbp product. The PCR extension time of 100 seconds was biased towards amplification of PCR products shorter than 6 kbp. The PCR products were separated on a 1% agarose gel. Deletion PCR products were excised from the gel, purified using QIAquick Gel Extraction Kit (Qiagen) and analysed by sequencing using BigDye Terminator version 3.1 (Applied Biosystems). The reaction products were analysed on an Applied Biosystems 3730 Genetic Analyser.

Using the method described above, a single SSIIa deletion mutant was identified. PCR analysis indicated the deletion was of approximately 3 kbps and DNA sequencing found the deletion occurred between base pairs 1166 and 3934 of the SSIIa gene. This single plant was grown to maturity. Visual inspection of the seeds from this plant found they displayed a shrunken phenotype while biochemical analysis found the starch content was reduced and the seeds lacked SSIIa enzyme activity. Transcripts of the SSIIa gene were reduced in length relative to wild type SSIIa as assayed by RT-PCR.

Isolation of a Rice SSIIa cDNA Clone and Production of a Suitable Plant Protein Expression Construct

A cDNA library of 2×10⁶ pfu was constructed from 25 μg of developing endosperm total RNA isolated from the of rice variety Vialone Nano (starch gelatinization temperature 62° C.) using a Stratagene cDNA synthesis kit, according to the manufacturer's instructions. Plaques harbouring SSIIa cDNA were identified by screening the library with a ³²P-labeled SSIIa gene PCR fragment. Positive plaques were identified and the pBluescript phagemids were excised from the Uni-ZAP XR vector. DNA sequence analysis of the excised phagemids identified a clone (pSCUSSIIa1) which harboured a full length SSIIa cDNA which was free of cloning artifacts. This clone was used in subsequent analysis.

The EMU promoter of the plasmid pEMUGN (Last et al., 1991) which consists of the EMU promoter and gus (uidA) gene and nos termination sequence, was excised by restriction digestion. The linearised plasmid was blunt ended and the maize ubiquitin gene (Ubi) promoter derived from pAHC18 (Bruce et al., 1989) inserted in its place. The SSIIa cDNA was excised from pSCUSSIIa and inserted 3′ to the UBI promoter and 5′ of gus gene of the modified pEMUGN plasmid to create the expression construct pSCUSSIIaEx1.

Transformation of the Target Rice Variety Basmati 370 with the Expression Construct pSCUSSIIaEx1

Transformation of the rice variety Basmati 370 with the expression construct pSCUSSIIaEx1 was achieved following the protocol of Abedinia et al. (1997). Briefly, dehulled seeds (caryopsis) were sterilized by rinsing in 70% ethanol followed by washing in 2% sodium hypochlorite and then sterile water. Callus induction medium (MSC), regeneration medium and medium for plantlet growth were as described in Abedinia et al. (1997). Culture plates were incubated in growth cabinet with 14 hour days at 27° C. and 10 hour nights of 22° C. A Dupont Biolistics PDS-1000/He (Bio-Rad) was utilised to transform the rice callus. The gold particles were coated with plasmid DNA according to Sanford et al. (1993) and bombardment conditions optimized using transient gus reporter gene assays (Jefferson, 1987). Plantlets of 10-15 cm were transplanted and transferred to a glasshouse.

Genomic DNA was extracted using a Qiagen Dneasy® 96 Plant Kit (Qiagen GMbH, Germany). Transformed plants were identified by PCR screening using primers for the gus gene as described in Abedinia et al. (1997) using the primers GUS-1 and GUS-2:

| | |
|---|---|
| GUS-1 5′-GGTTGGGCAGGCCAGCGTATC-3′ | (SEQ ID NO:2) |
| GUS-2 5′-CCAATGCCTAAAGAGAGGTTA-3′ | (SEQ ID NO:3) |

and for the inserted SSIIa cDNA using the primers SSIIaF1 and SSIIaR1:

| | |
|---|---|
| SSIIaF1 5′-GTCGCTCGCTGCCCATT-3′ | (SEQ ID NO:4) |
| SSIIaR1 5′-TGCCCCTCCGCTTCAA-3′ | (SEQ ID NO:5) |

which generated a 495 bp PCR amplicon specific to the SSIIa cDNA.

Southern hybridization analysis was used to determine the integration pattern of SSIIa. Genomic DNA was digested with EcoRV and transferred onto Hybond N membrane (Amersham) according to the manufacturer's instructions. A ³²P-labeled PCR product generated by the primers SSIIaF1 and SSIIaR1 was used as a probe for hybridization. A plant harbouring a single insertion of the Vialone Nano SSIIa cDNA was identified. This plant was grown to maturity, the seeds harvested and Ti generation plants backcrossed to Basmati 370. Analysis of the starch of F2 plants resulted in the identification of plants which produced seed with a starch gelatinization temperature that was significantly reduced relative to the parental strain, Basmati 370.

The foregoing embodiments are illustrative only of the principles of the invention, and various modifications and changes will readily occur to those skilled in the art. The invention is capable of being practiced and carried out in various ways and in other embodiments. It is also to be understood that the terminology employed herein is for the purpose of description and should not be regarded as limiting.

The term “comprise” and variants of the term such as “comprises” or “comprising” are used herein to denote the inclusion of a stated integer or stated integers but not to exclude any other integer or any other integers, unless in the context or usage an exclusive interpretation of the term is required.

Any reference to publications cited in this specification is not an admission that the disclosures constitute common general knowledge in Australia.

### REFERENCES

Abedinia, M., Henry, R. J., Blakeney, A. B. and Lewin, L. (1997) An efficient transformation system for the Australian rice cultivar, Jarrah. Australian Journal of Plant Physiology. 24, 133-141
Abremski, K., Hoess R., Steniberg, N. (1983) Studies on the properties of P1 site-specific recombination: evidence for topologically unlinked products following recombination. Cell. 32(4):1301-11.
Adelman, J. P., Hayflick, J. S., Vasser, M. and Seeburg, P. H. (1983) In vitro deletional mutagenesis for bacterial production of the 20,000-dalton form of human pituitary growth hormone. DNA 2: 183-193
Angell, S. M., and Baulcombe, D. C. (1997). Consistent gene silencing in transgenic plants expressing a replicating potato virus X RNA. EMBO J. 16, 3675-3684.
Bartley, G. E., Viitanen, P. V., Bacot, K. O., Scolnik, P. A. (1992) A tomato gene expressed during fruit ripening encodes an enzyme of the carotenoid bio-synthesis pathway. Journal of Biological Chemistry 267: 5036-5039
Barton and Chilton (1983) Agrobacterium Ti plasmid vectors for plant genetic engineering. Methods in Enzymology 101: 527-539
Bennetzen, J. L. (1996) The contributions of retroelements to plant genome organization, function and evolution. Trends Microbiol 4: 347-353
Bernstein, E., Denli, A. M., and Hannon, G. J. (2002) The rest is silence. RNA 7: 1509-1521
Bird, A. R., Flory, C., Davies, D. A., Usher, S, and Topping, D. L. (2004) A novel barley cultivar (Himalaya 292) with a specific gene mutation in starch synthase 11a raises large bowel starch and short-chain fatty acids in rats. Journal of Nutrition 134, 831-835.
Bird et al. (1991), Bio/Technology 9: 635-639
Bruce, W. B., Christensen, A. H., Klein, T., Fromm, M., and Quail, P. H. (1989) Photoregulation of a phytochrome gene promoter from oat transferred into rice by particle bombardment. Proceedings of the National Academy of the Sciences 86, 9692-9696
Brummelkamp (2002) A system for stable expression of short interfering RNAs in mammalian cells Science 296: 550-553
Carter P., Bedouelle H., and Winter G. (1985) Improved oligonucleotide site-directed mutagenesis using M13 vectors. Nucleic Acids Res. 13(12):4431-43
Craig, J., Lloyd, J. R., Tomlinson, K., Barber, L., Edwards, A., Wang, T. L., Martin, C., Hedley, C. L. and Smith, A. M. (1998) Mutations in the gene encoding starch synthase II profoundly alter amylopectin structure in pea embryos. Plant Cell 10, 413-426.
Cunningham, B. C. and Wells, J. A., (1989) High-resolution epitope mapping of hGH-receptor interactions by alanine-scanning mutagenesis. Science 244, 1081-1085.
Ding, S. W. (2000), RNA silencing Current Opinion in Biotechnology 11: 152-156.
Duan X. L., Li X. G., Xue Q. Z., AboElSaad M., Xu D. P., and Wu, R. (1996) Transgenic rice plants harboring an introduced potato proteinase inhibitor II gene are insect resistant. Nature Biotechnology 14: 494-498
Fitzgerald, M. (2004) Starch. In: Rice: Chemistry and Technology, 3rd ed. Edited by Elaine T. Champagne. AACC, St Paul, Minn., USA. pp 109-141.
Geierson and Corey, Plant Molecular Biology, 2d Ed. (1988)
Gelvin, S. B. (2000) Agrobacterium and plant genes involved in T-DNA transfer and integration. Annual Review of Plant Physiology Plant Molecular Biology 51: 223-256
George, E. F. (1993)Plant propagation by Tissue Culture Part 1, Energetics Limited, Edington pp 574.
Glick and Thompson (eds.), Methods in Plant Molecular Biology and Biotechnology, Boca Raton, Fla.: CRC Press (1993)
Gray, J., Pictor, S., Shabbeer, J., Schuch, W., and Grierson, D. (1992). Plant Molecular Biology 19:69-87.
Greco, R., Ouwerkerk, P. B. F., Sallaud, C., Kohli, A., Colombo, L., Puigdomenech, P., Guiderdoni, E., Christou, P., Hoge, J. H. C., and Pereira, A. (2001) Transposon insertional mutagenesis in rice. Plant Physiology, 125, 1175-1177.
Grierson, D., Fray, R. G., Hamilton, A. J., Smith, C. J. S., and Watson, C. F. (1991). Does co-suppression of sense genes in transgenic plants involve antisense RNA. Trends Biotech. 9, 122-123.
Hamilton, A. J. and Baulcombe, D. C. A Species of Small Antisense RNA in Posttranscriptional Gene Silencing in Plants. Science 286(5441) 950-952 (1999)
Hannon (2002) RNA Interference, Nature 418: 244-251
Henikoff, S, and Comai, L. (2003) Single-nucleotide mutations for plant functional genomics. Annual Review of Plant Biology 54: 375-401
Andrew Hiatt (Ed) (1993). Transgenic Plants: Fundamentals and Applications, Marcel Dekker NY.
Hirochika, H., Guiderdoni, E., An. G., Hsing. Y. I., Eun, M. Y., Han, C. D., Upadhyaya, N., Ramachandran, S., Zhang, Q., Pereira, A., Sundaresan, V., Leung, H. (2004) Rice mutant resources for gene discovery. Plant Mol. Biol. 54(3):325-34.
Hirochika, H., Guiderdoni, E., An, G., Hsing, Y. I., Eun, M. Y., Upadhyaya, N. M., Ramachandran, S., Zhang, Q., Pereira, A., Sundaresan, V., Leung, H. (2004) Rice mutant resources for gene discovery. Plant Molecular Biology 54(3):325-334
Hirochika, H., Sugimoto, K., Otsuki, Y., Tsugawa, H., Kanda, M. (1996) Retrotransposons of Rice Involved in Mutations Induced by Tissue Culture Proc Natl Acad Sci USA. 93(15): 7783-7788
Hunter, C. P. (1999) Genetics: A touch of elegance with RNAi. Current Biology 9 (12): R440-R442
Hutvagner, G., Zamore, P. D. (2002) RNAi: nature abhors a double-strand. Current Opinion in Genetics and Development. 12, 225-232.
Jefferson, R. A. (1987) Assaying chimeric genes in plants: the GUS gene fusion system. Plant Molecular Biology Reporter 5, 387-405
Jiang, H. W., Dian, W. M., Liu, F. Y., Wu, P. (2004) Molecular cloning and expression analysis of three genes encoding starch synthase II in rice. Planta 218, 1062-1070.
Kim, S. H., Cech, T. R. (1987) Three-dimensional model of the active site of the self-splicing rRNA precursor of Tetrahymena. Proceedings of the National Academy of the Sciences USA 84: 8788-8792
Kipp, P. B., Van Eck, J. M., Beetham, P. R., May, G. D. (1999) “Gene targeting in plants via site-directed mutagenesis” in Gene Targeting Protocols (Kmiec, E. B., ed.), Humana Press, Totowa, N. J., pp. 213-221
Kristensen, V. N., Kelefiotis, D., Kristensen, T., Borresen-Dale A. L. (2001) High-throughput methods for detection of genetic variation. Biotechniques, 30(2):318-22, 324, 326 passim.
Krysan. P. J., Young, J. C., Jester, P. J., Monson, S., Copenhaver, G., Preuss, D., Sussman, M. R. (2002) Characterization of T-DNA insertion sites in Arabidopsis thaliana and the implications for saturation mutagenesis. OMICS 6:163-174
Last, D. I., Brettell, R. I. S., Chamberlain, D. A., Chaudhury, A. M., Larkin, P. J., Marsh, E. L., Peacock, W. J., Dennis, E. S. (1991) The use of the Emu promoter with antibiotic and herbicide resistance genes for the selection of transgenic wheat callus and rice plants. Theorectical and Applied Genetics 81, 581-588
Leszek, A., Lyznik, K. V., Rao and Thomas K. Hodges (1996) FLP-mediated rearrangements of FRT sites in the maize genome. Nucleic Acids Research 24:3784-3789.
Leung, D W., Chen, E., Goeddel, D. V. (1989) A method for random mutagenesis of a defined DNA segment using a modified polymerase chain reaction. Technique 1, 11-15.
Li, X., Song, Y., Century, K., Straight, S., Ronald, P., Dong, X., Lassner, M., Zhang, Y. (2001) A fast neutron deletion mutagenesis-based reverse genetics system for plants Plant Journal 27(3):235-242
Luo, H., Lyznik, L. A., Gidoni, D., Hodges, T. K. (2000) FLP-mediated recombination for use in hybrid plant production. The Plant Journal 23(3):423-30.
McCallum, C. M., Comai, L., Greene, E. A., Henikoff, S. Targeted screening for induced mutations. (2000) Nature Biotechnology 18:455-457
Mayers et al. (1985), Science 229:242
Methods in Molecular Biology (1997) vol. 74, Chapter 43 Expressing Ribozymes in Plants (Turner and Humana Press, Totowa, N.J., Eds.)
Miki and Iyer, Fundamentals of Gene Transfer in Plants. In: Plant Metabolism, 2nd ed., Dennis, D. T., Turpin, D. H., Lefebrve, D. D., Layzell, D. B. (eds), Addison Wesly, Langmans Ltd. London. pp. 561-579 (1997)
Mol, J. N., van der Krol, A. R., van Tunen, A. J., van Blokland, R., de Lange, P., Stuitje, A. R. (1990) Regulation of plant gene expression by antisense RNA. FEBS Lett. 268(2):427-30
Morell, M. K., Kosar-Hashemi, B., Cmiel, M., Samuel, M. S., Chandler, P., Rahman, S., Buleon, A., Batey, I. L., Li, Z. Y. (2003) Barley sex6 mutants lack starch synthase Ia activity and contain a starch with novel properties. Plant Journal 34, 172-184.
Napoli, C., Lemieux, C., and Jorgensen, R. (1990). Introduction of a chimeric chalcone synthase gene into petunia results in reversible co-suppression of homologous genes in trans. Plant Cell 2, 279-289.
Panlasigui, L. N., Thompson, L. U., Juliano, B. O., Perez, C. M., Yiu, S. H., Greenberg, G. R. (1991) Rice varieties with similar amylose content differ in starch digestibility and glycemic response in humans. Am J Clin Nutr. 54, 871-7.
Reinhold-Hurek, B., Shub, D. A. (1992) Self-splicing introns in tRNA genes of widely divergent bacteria. Nature 357:173-176.
Rothstein, S. J., DiMaio, J., Strand, M., Rice, D. 1987. Stable and inheritable inhibition of the expression of nopaline synthase in tobacco expressing antisense RNA. Proc. Natl. Acad. Sci. USA 84: 8439-8443
Sambrook et al. (eds.), Molecular Cloning: A Laboratory Manual (Second Edition), Plainview, N.Y. Cold Spring Harbor Laboratory Press (1989)
Sanford, J. C., Smith, F. D., Russell, J. A. (1993) Optimizing the biolistic process from different biological applications. Methods in Enzymology 217, 483-509
Smith, C. J., Watson, C. F., Ray, J., Bird, C. R., Morris, P. C., Schuch, W., Grierson, D. (1988) Antisense RNA inhibition of polygalacturonase genes in transgenic tomatoes. Nature 334, 724-726
Sternberg, N. (1981). Bacteriophage P1 site-specific recombination. III. Strand exchange during recombination at lox sites. J. Mol. Biol. 150: 603-608.
Ugaki, M., Ueda, T., Timmermans, M. C. P., Vieira, J., Elliston, K. O., Messing, J. (1991) Nucleic Acids Res., 19, 371-377.
Umemoto, T., Aoki, N., Lin, H. X., Nakamura, Y., Inouchi, N., Sato, Y., Yano, M., Hirabayashi, H., Maruyama, S. (2004) Natural variation in rice starch synthase Ia affects enzyme and starch properties. Functional Plant Biology 31, 671-684.
Umemoto, T., Yano, M., Satoh, H., Shomura, A., Nakamura, Y. (2002) Mapping of a gene responsible for the difference in amylopectin structure between japonica-type and indica-type rice varieties. Theoretical and Applied Genetics 104, 1-8.
van der Krol, A., Lenting, P. E., Veenstra, J., van der Meer, I. M., Koes, R. E., Gerats, A. G. M., Mol, J. N. M., Stuitje, A. R. (1988) An anti-sense chalcone synthase gene in transgenic plants inhibits flower pigmentation. Nature 333, 866-869.
van der Krol, A. R., Mol, J. N. M., and Stuitje, A. R. (1988) Modulation of eukaryotic gene expression by complementary RNA or DNA sequences. Biotechniques; 6:958-976.
van der Krol, A. R., Mur, L. A., Beld, M., Mol, J. N. M., Stuitje, A. R. (1990). Flavonoid genes in petunia: Addition of a limited number of gene copies may lead to a suppression of gene expression. Plant Cell 2, 291-299.
Wells, J. A., Vasser, M., and Powers, D. B. (1985) Cassette mutagenesis: an efficient method for generation of multiple mutations at defined sites. Gene.; 34(2-3): 315-23.
Wells, J. A., Cumningham, B. C., Graycar, T. P., and Estell, D. A. (1986) Importance of hydrogen-bond formation in stabilizing the transition state of subtilisin. Phil. Trans. R. Soc. Lond. A 317, 415-423
Weissbach and Weissbach, Methods for Plant Molecular Biology, Academic Press, New York VIII, pp. 421-463 (1988)
Wright, D. A., Ke, N., Smalle, J., Hauge, B. M., Goodman, H. M., and Voytas, D. F. (1996) Multiple Non-LTR Retrotransposons in the Genome of Arabidopsis thaliana. Genetics 142: 569-578.
Zhu, T., Mettenburg, K., Peterson, D. J., Tagilani, L., and Baszczynski, C. L. (2000), Nature Biotechnology 18, 555-561
Zhu, T., Peterson, D. J., Tagliani, L., St. Clair, G., Baszczynski, C. L., and Bowen, B. (1999) Proc. Natl. Acad. Sci. USA, 96, 8774-8778
Ziemienowizc, A. (2001) Plant selectable markers and reporter genes Acta Physiologiae Plantarum, 23(3):363-374
Zoller, M. J., and Smith, M. (1982) Oligonucleotide-directed mutagenesis using M13-derived vectors: an efficient and general procedure for the production of point mutations in any fragment of DNA. Nucleic Acids Res. 10(20):6487-6500)

1-32. (canceled)

33. A process for producing a modified plant comprising starch with an altered gelatinization temperature relative to the gelatinization temperature of starch of a parental strain of said plant, the process comprising the steps of:
i) obtaining tissue from a parental plant strain;
ii) changing the starch synthase IIa gene of said tissue so that starch synthesized by enzymes including starch synthase IIa encoded by the changed gene have an altered gelatinization temperature; and
iii) propagating plants from the tissue prepared in step (ii);
wherein
said changing the starch synthase IIa gene in step (ii) comprises altering nucleotides of said gene of the parental plant strain, and wherein said alteration is selected from the group consisting of:
in the motif G(LV)RDTV, the second V is replaced by any other hydrophobic residue; and
in the motif (EK)SW(RKE) (AGS)L, the L is replaced by any other hydrophobic residue.

34. The process of claim 33, wherein said tissue obtained in step (ii) is selected from the group consisting of seeds, roots, leaves and stems.

35. The process of claim 33, wherein in the motif G(LV)RDTV, the second V is replaced by M.

36. The process of claim 33, wherein in the motif (EK)SW(RKE) (AGS)L, the L is replaced by any aromatic residue.

37. The process of claim 36, wherein in the motif (EK)SW(RKE) (AGS)L, the L is replaced by F.

38. A modified plant comprising starch with an altered gelatinization temperature relative to the gelatinization temperature of starch of a parental strain of said plant, wherein altered gelatinization temperature results from changing the starch synthase IIa gene of said parental strain by altering nucleotides of said gene, and wherein said alteration is selected from the group consisting of:
in the motif C(LV)RDTV, the second V is replaced by any other hydrophobic residue; and
in the motif (EK)SW(RKE) (AGS)L, the L is replaced by any other hydrophobic residue.

39. The plant of claim 38, being a monocotyledonous or dicotyledonous plant.

40. The plant of claim 38, being a cereal crop plant selected from the group consisting of rice, oats, barley, sorghum, maize, wheat, rye, amaranth, rape, and spelt.

41. The plant of claim 38, selected from the group consisting of potato and taro.

42. The plant of claim 38, being a legume selected from the group consisting of alfafa, beans, broom, carob, clover, cowpea, mung bean, mimosa, peas, peanuts, soybeans, tamarind, and vetch.

43. The plant of claim 38, wherein in the motif G(LV)RDTV, the second V is replaced by M.

44. The plant of claim 38, wherein in the motif (PK)SW(RKE) (AGS)L, the L is replaced by any aromatic residue.

45. The plant of claim 44, wherein in the motif (EK)SW(RKE) (AGS)L, the L is replaced by F.

46. A product comprising starch obtained from the modified plant of claim 38.

47. The product of claim 46, selected from the group consisting of rice, pasta, bread, noodles, and potato.

48. A process for producing a modified starch synthase IIa gene so that starch synthesized by enzymes including the starch synthase IIa encoded by said modified gene have an altered gelatinization temperature, the process comprising the steps of:
i) identifying variations in starch synthase IIa genes of strains of plants;
ii) correlating a desired gelatinization temperature with specific changes in said genes; and
iii) making said specific changes in the starch synthase IIa gene of a subject plant to obtain said modified gene.

49. A modified starch synthase IIa gene product of the process according to claim 48.

50. A modified starch synthase IIa gene, wherein said gene encodes any one or any combination of the following changes relative to the starch synthase IIa gene of a parental plant strain:
in the motif G(LV)RDTV, the second V is replaced by any other hydrophobic residue; and
in the motif (EK)SW(RKE) (AGS)L, the L is replaced by any other hydrophobic residue.

51. The modified starch synthase IIa gene of claim 50, wherein in the motif G(LV)RDTV, the second V is replaced by M.

52. The modified starch synthase IIa gene of claim 50, wherein in the motif (EK)SW(RKE) (AGS)L, the L is replaced by any aromatic residue.

53. The modified starch synthase IIa gene of claim 52, wherein in the motif (EK)SW(RKE) (AGS)L, the L is replaced by F.

54. A process for producing starch with a selected gelatinization temperature, the process comprising the steps of:
i) obtaining tissue from a plant to be used as a source of starch with said selected gelatinization temperature;
ii) changing the starch synthase IIa gene of said tissue so that starch synthesized by enzymes including starch synthase IIa encoded by the changed gene have said selected gelatinization temperature;
iii) propagating plants from the tissue prepared in step (ii); and
iv) harvesting starch from said plants propagated in step (iii).

55. A starch product of the process according to claim 54.

56. The starch product of claim 55, selected from the group consisting of rice, pasta, bread, noodles, and potato.
